(19) United States
(12) Patent Application Publication
(10) Pub. No.: US 2009/0210008 A1
(43) Pub. Date: Aug. 20, 2009

(54) Modular spine plate with projection and socket interface

(75) Inventors: Michael S. Butler, St. Charles, IL (US); Michael J. Milella, JR., Schaumburg, IL (US)

Correspondence Address: BOWMAN & ASSOCIATES, 1016 3rd Avenue, SW, Suite #106, CARMEL, IN, 46032, US

(73) Assignee: Life Spine, Inc.

(21) Appl. No.: 12/070,683

(22) Filed: Feb. 20, 2008

(51) Int. Cl. A61B 17/70

(52) U.S. Cl. 606/280; 606/70

(57) ABSTRACT

A modular spine plate is formed of two or more spine plate components that connect to one another through projection and socket interfaces. The projection and socket interfaces may provide for snap fit features that allow locking connectivity between the spine plate components. The modular spine plate components constitute a single end plate component and a single center or middle plate component. The end plate component has a first end plate leg and a second end plate leg. One of the first and second end plate legs includes an end socket while the other of the first and second end plate legs includes an end projection sized to be received in the end socket. The center plate component has a first center plate leg, a second center plate leg, a third center plate leg, and a fourth center plate leg. One of the first and second center plate legs includes a center socket while the other of the first and second center plate legs includes a center projection. To create N-level modular spine components, end plate components may be joined to one another through 180° rotation, center plate components may be joined with end plate components through 180° rotation, and center plate components may be joined with center plate components through 180° rotation with end plate components through 180° rotation.

### BACKGROUND OF THE INVENTION

1. Field of the Invention

The present invention relates generally to devices for the internal fixation of the spine such as spinal implants for holding vertebral bones fixed relative to one another and, more particularly, to static bone fixation implants for use in spinal surgical procedures for stabilizing the relative motion thereof by temporarily or permanently immobilizing bones of the spine.

2. Background Information

Spine plates have been used for many years to increase spine stability following single and multi-level spine surgery. Particularly, spine plates implanted during surgery for reasons such as disease, trauma, defect, accident or the like, are used to stabilize one or more spinal vertebrae. Stabilization leads to a proper healing or a desired outcome.

In some instances, it is desirous to cause the fusion of two adjacent vertebrae. If this is the case, the surgeon makes an incision to reach the spine. Tissues and muscles are retracted (spread apart) to reveal the proper level in the spine. The cartilaginous material or disc between the two vertebrae is removed and the bone surface abraded to encourage a bleeding surface. Blood from the bleeding surfaces is desired in order for the bones to fuse. The space between the adjacent vertebrae is filled with bone graft.

The spine plate is mounted to two or more vertebrae during the surgery. Bone screws are used to mount the spine plate to the one or more vertebrae. It is important during the mounting process that the spine plate be properly aligned on the vertebrae for receipt of the mounting screws. In all cases, the spine plate must be fastened onto the superior (top) and inferior (bottom) of the vertebra via bone screws. This stabilizes the spine to facilitate fusion and healing. The bone screws are received in bores of the spine plate and hold the spine plate to the vertebra.

Because the anatomy of each patient is different, various lengths of spine plates must be manufactured in order to accommodate these variations. Also, spine plates are manufactured having various levels in order to accommodate the fixation of more than two vertebrae. For instance, a spine plate that attaches to and fixes two adjacent vertebrae is known as a one level (1-L) spine plate. The 1-L spine plate thus spans only a single spinal disc area. A spine plate that attaches to and fixes three adjacent vertebrae is known as a two level (2-L) spine plate. The 2-L spine plate thus spans two spinal disc areas. Moreover, a spine plate that attaches to and fixes four adjacent vertebrae is known as a three level (3-L) spine plate. The 3-L spine plate thus spans three spinal disc areas. Spine plates of additional levels may also be made. When multi-level spine plates are taken into account for patient variation, a vast inventory of spine plates of various lengths must be maintained.

It would thus be desirable to limit the inventory of spine plates. It would be further desirable to have a spine plate that utilizes a minimum of components to achieve anywhere from a 1-L to a multi-level spine plate. It would also be desirable to provide the desired minimum number of components in a modular format of spine plate. Still further, it would be desirable that the various components of a modular spine plate be easily and securely assembled.

### SUMMARY OF THE INVENTION

A modular spine plate is formed of two or more plate components that connect to one another through projection and socket interfaces. The modular spine plate components constitute a single end plate component and a single center or middle plate component.

The end plate component has a first end plate leg extending from an upper portion of a first side thereof and a second end plate leg extending from a lower portion of the first side. One of the first and second end plate legs includes or defines an end socket while the other of the first and second end plate legs includes or defines an end projection sized to be received in the end socket. The end plate leg having/defining an end socket may just be deemed an end socket. The end plate leg having/defining an end projection may just be deemed an end projection. The end projection includes a resilient flange while the end socket includes a slot. The end component also includes an upper bone screw bore and a lower bone screw bore that are each adapted to receive a bone screw for attaching the end plate component to a vertebra.

In one form, a one level (1-L) modular spine plate is formed of two of the end plate components one of which is turned 180° relative to the other end plate component. The end projection of the first end plate component is received in the end socket of the second end plate component while the end projection of the second end component is received in the end socket of the first end component. The resilient flanges of each end projection are received in the socket slots of each end socket to provide a snap fit of the end projections into the end sockets thereby locking the two end plate components together to form the 1-L modular spine plate from two modular spine plate components. The socket slots may be sized to accommodate movement of the resilient flanges so as to allow limited movement of the two end plate components relative to one another.

The center plate component has a first center plate leg extending from an upper or first portion of a first side thereof, a second center plate leg extending from a lower or second portion of the first side, a third center plate leg extending from an upper or third portion of a second side thereof, and a fourth center plate leg extending from a lower or fourth portion of the second side. The center plate component includes an upper bone screw bore and a lower bone screw bore that are each adapted to receive a bone screw for attaching the center plate component to a vertebra. One of the first and second center plate legs includes a center socket while the other of the first and second center plate legs includes a center projection. One of the third and fourth center plate legs includes a center projection while the other of the third and fourth center plate legs includes a center socket. The center plate legs having/defining a center socket may just be deemed center sockets. The center plate legs having/defining a center projection may just be deemed center projections. The center projections each include a resilient flange while the center sockets each include a slot. The center projections and the center sockets are disposed diagonally opposite one another on their respective side.

In one form, a two-level (2-L) modular spine plate is formed of a center component and two end plate components. The end projection of the first end plate component is received in the first center socket on the first side of the center plate component while the end socket of the end projection of the first end plate component receives the first center projection on the first side of the center plate component. The resilient flanges of the end projections are received in the socket slots of each end socket to provide a snap fit of the end projections into the end sockets thereby locking the first end plate component to the center plate component. The end projection of the second end plate component is received in the second center socket on the second side of the center plate component while the end socket of the end projection of the second end plate component receives the second center projection of the second side of the center plate component. The resilient flanges of the end projections are received in the socket slots of each end socket to provide a snap fit of the end projections into the end sockets thereby locking the second end plate component to the center plate component. In this manner, a 2-L modular spine plate is formed using two modular spine plate components. Again, the socket slots may be sized to accommodate movement of the resilient flanges so as to allow limited movement of the two end plate components relative to one another.

In one form, a three level (3-L) modular spine plate is formed of two center plate components and two end plate components. The two center plate components are snap fitted together in the manner described above wherein one of which is turned 180° relative to the other center plate component, while an end plate component is snap fitted onto each center plate component in the manner described above. It can be seen that multi-level modular spine plates may be formed greater than three levels by adding additional center plate components wherein adjacent center plate components are turned 180° relative to one another.

As well, in another form of the invention, there is provided a kit for assembling an N-level modular spine plate. The kit includes one or more middle plate components and two end plate components.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a top plan view of an exemplary embodiment of a one level (1-L) modular spine plate fashioned in accordance with the present principles;

FIG. 2 is a side view of the 1-L spine plate of FIG. 1 taken along line 2-2 thereof;

FIG. 3 is a top plan view of an exemplary embodiment of a two level (2-L) modular spine plate fashioned in accordance with the present principles;

FIG. 4 is a side view of the 2-L spine plate of FIG. 3 taken along line 4-4 thereof;

FIG. 5 is a top plan view of a 3-L modular spine plate fashioned in accordance with the present principles;

FIG. 6 is a side view of the 3-L spine plate of FIG. 5 taken along line 6-6 thereof;

FIG. 7 is an enlarged top plan view of an end component of the modular spine plates of FIGS. 1-6 fashioned in accordance with the present principles;

FIG. 8 is an enlarged bottom plan view of the end component of FIG. 7;

FIG. 9 is an end view of a portion of the end component of FIG. 7 taken along line 9-9 thereof particularly showing an end component socket thereof;

FIG. 10 is an enlarged sectional view of the end component as shown in FIG. 8 taken along line 10-10 thereof;

FIG. 11 is an enlarged portion of the sectional view of FIG. 10 taken along circle 11-11 thereof;

FIG. 12 is an enlarged top plan view of a center component of the modular spine plates of FIGS. 3-6 fashioned in accordance with the present principles;

FIG. 13 is an enlarged bottom plan view of the center component of FIG. 12; and

FIG. 14 is an enlarged sectional view of the end component of FIGS. 12 and 13.

Like reference numerals indicate the same or similar parts throughout the several figures.

A description of the features, function and/or configuration of the components depicted in the various figures will now be presented. It should be appreciated that not all of the features of the components of the figures are necessarily described. Some of these non discussed features are inherent from the figures. Other non discussed features may be inherent in component geometry and/or configuration.

### DETAILED DESCRIPTION OF THE PREFERRED EMBODIMENTS

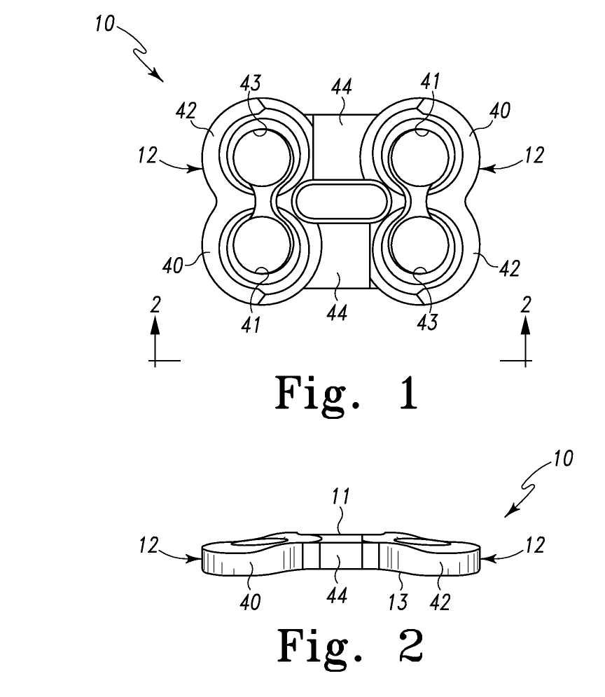

Referring to the figures and particularly to FIGS. 1 and 2, there is depicted an embodiment of a single level (1-L) modular spine plate generally designated 10 fashioned in accordance with the present principles. It should be understood that the 1-L spine plate 10 is representative of various styles of modular spine plates such as those that utilize lock tabs for bone screws (not shown) and those that do, as well as other styles. The 1-L modular spine plate 10 is formed of a suitable biocompatible material such as titanium, stainless steel, alloys thereof, and other biomaterials. As best discerned in FIG. 2 the modular 1-L spine plate 10 is curved or arched from end to end (along a saggittal plane). The modular 1-L spine plate 10 is comprised of two end plate components 12 defining a posterior side 13 and an anterior side 11. The end plate component 12 may be made in various sizes.

Particularly, the 1-L modular spine plate 10 is formed of two identical end plate components 12 one of which is rotated or turned 180° relative to the other. As described in greater detail below, the end plate components are joined, attached or connected to one another via a projection and socket interface. The projection and socket interface also includes a flange and slot snap fit feature.

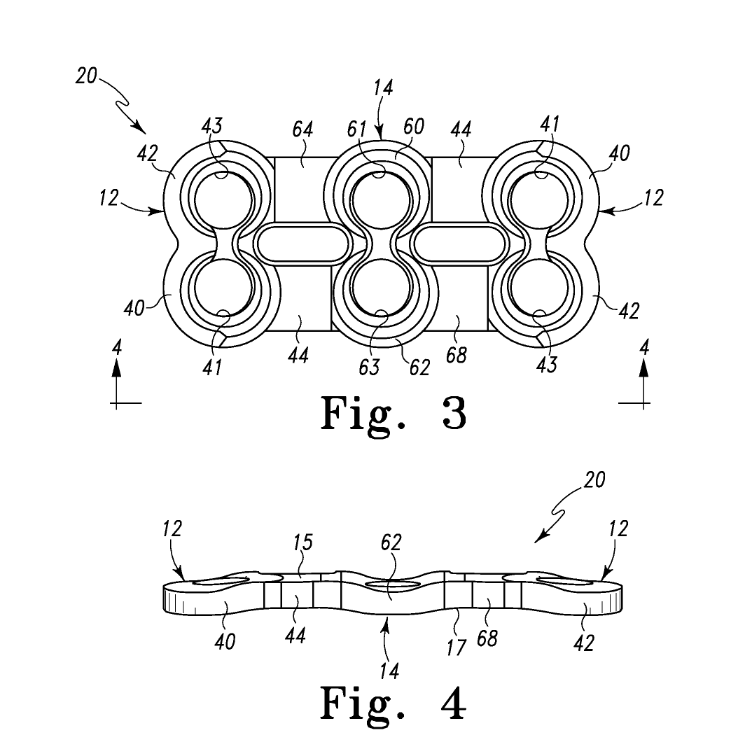

FIGS. 3 and 4 depict an embodiment of a two level (2-L) modular spine plate generally designated 20 fashioned in accordance with the present principles. It should be understood that the modular 2-L spine plate 20 is representative of various styles of modular spine plates such as those that utilize lock tabs for bone screws (not shown) and those that do, as well as other styles. The 2-L modular spine plate 20 is formed of a suitable biocompatible material such as titanium, stainless steel, alloys thereof, and other biomaterials. Particularly, the 2-L modular spine plate 20 is formed of two identical end plate components 12 and a center or middle plate component 14 defining a posterior side 16 and an anterior side 15. As best discerned in FIG. 4 the modular 2-L spine plate 20 is curved or arched from end to end (along a saggittal plane). The center plate component 14, like the end plate component 12, may be made in various sizes.

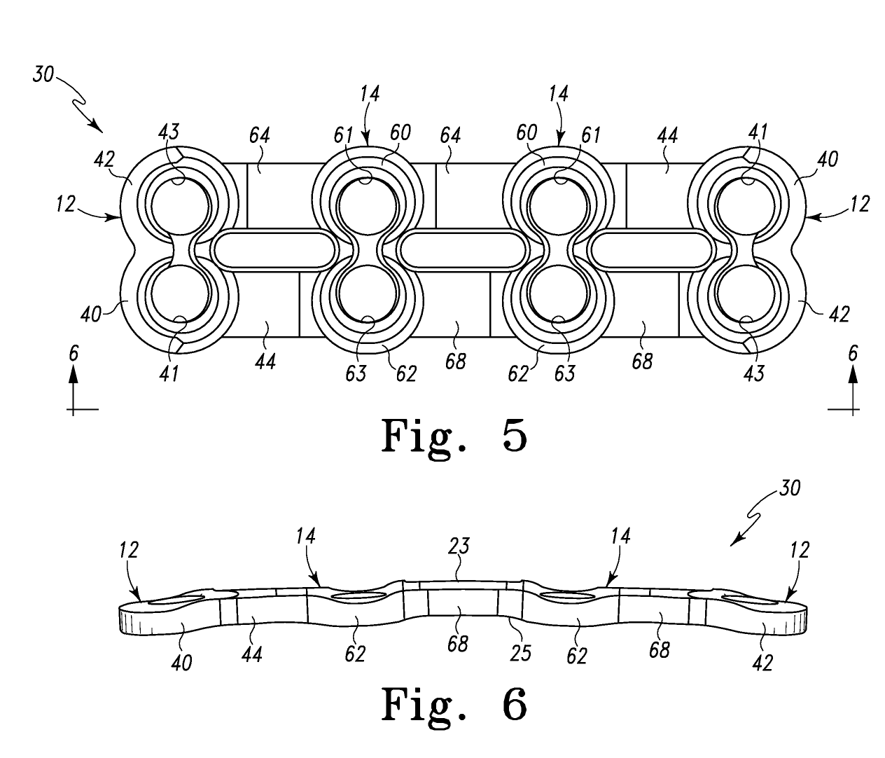

FIGS. 5 and 6 depict an embodiment of a three level (3-L) modular spine plate generally designated 30 fashioned in accordance with the present principles. It should be understood that the modular 3-L spine plate 30 is representative of various styles of modular spine plates such as those that utilize lock tabs for bone screws (not shown) and those that do, as well as other styles. The 3-L modular spine plate 30 is formed of a suitable biocompatible material such as titanium, stainless steel, alloys thereof, and other biomaterials. Particularly, the 3-L modular spine plate 30 is formed of two identical end plate components 12 and two identical center or middle plate components 14 defining a posterior side 24 and an anterior side 23. As best discerned in FIG. 6 the modular 2-L spine plate 20 is curved or arched from end to end (along a saggittal plane).

It should be appreciated that multi-level or N-level modular spine plates may be fashioned utilizing only these two plate components. Particularly, additional level modular spine plates (those in excess of three levels) are made by adding additional middle plate components.

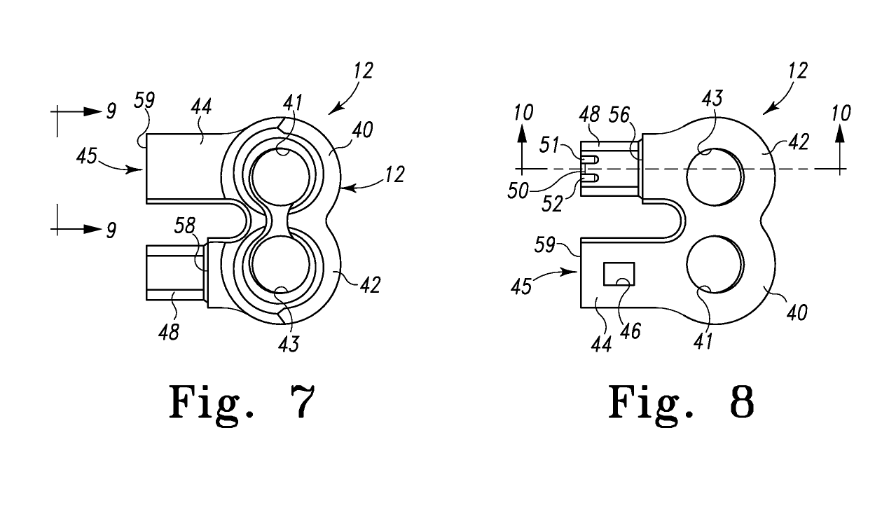

Referring to FIGS. 7-11 the end plate component 12 will be described. The end plate component 12 has a first boss 40 and a second boss 42. A bore 41 is provided in the first boss 40 and is configured to receive a bone screw (not shown) therethrough for attaching the end plate component 12 onto a vertebra. A bore 43 is provided in the second boss 42 and is configured to receive a bone screw (not shown) therethrough for attaching the end plate component 12 onto a vertebra. Extending from one side of the boss 40 is a leg 44 defining a socket 45. Extending from one side of the boss 42 is a leg 48 defining a projection. The legs 44 and 48 are on the same side of their respective bosses and provide an opening therebetween that defines a graft window.

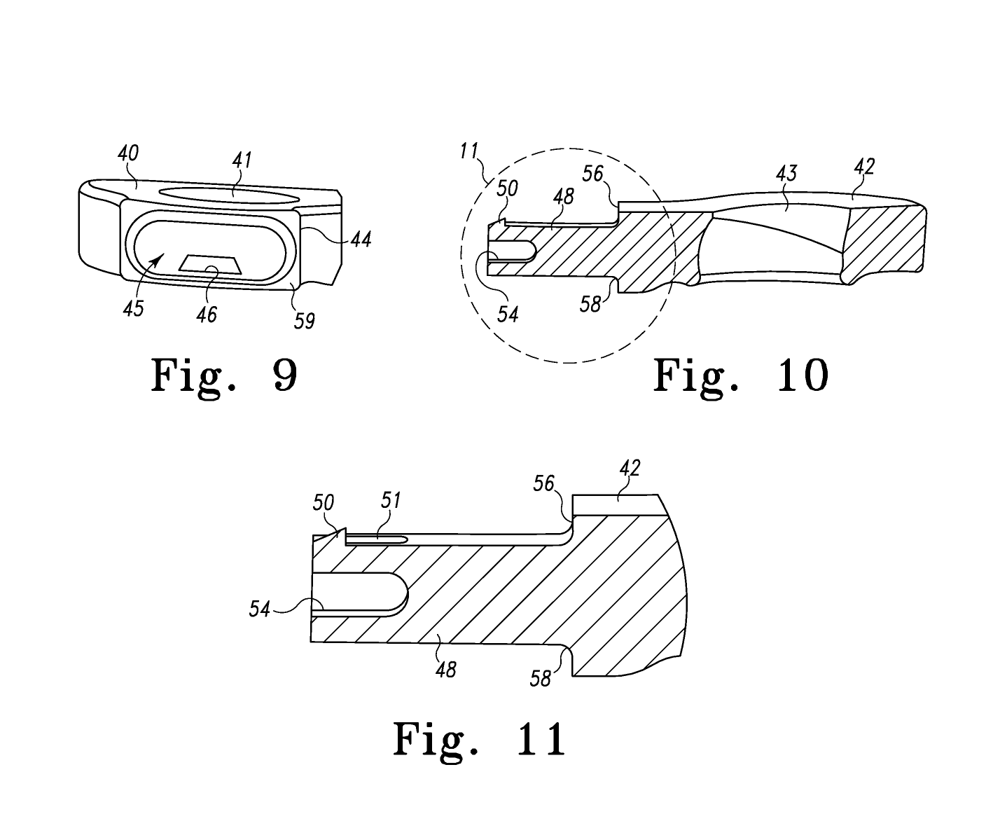

As best seen in FIG. 9, the socket 45 of the leg 44 is sized to receive the projection 48 of the end plate component 12 such that when a 1-L modular spine plate made of two identical end plate components 12 are joined, the projection 48 of one end plate component 12 is received in the socket 45 of another end plate component 12 and the projection 48 of the another end plate component 12 is received in the socket 45 of the one end plate component 12. The socket 45 also receives a projection (66 or 70) of the center or middle plate component 14 such as is described below when a 2-L or larger modular spine plate is formed.

As best seen in FIGS. 8, 10 and 11, the projection 48 has a resilient flange 50 on an end thereof formed in conjunction with side slots 51, 52 and middle slot 54. The slots 51, 52 aid in defining the flange 50. The flange 50 is resilient in that it deforms into the middle slot 54 when received in a socket of an end component 12 or center component 14. As seen in FIGS. 8 and 9, the leg 44 has an opening or slot 46 within and in communication with the socket 45. The flange 50 of the projection 48 is received in the slot 46 when an end plate component 12 is being engaged with an end plate component 12, or a flange 78 of projection 66 or flange 84 of projection 70 when the end plate component 12 is being engaged with a center plate component 14. The flange thus resiliently snaps into the socket to provide a snap fit and coupling of a projection with a socket. The surfaces 56 and 58 of the projection 48 abut a socket end (such as socket end 59 of an end plate component 12).

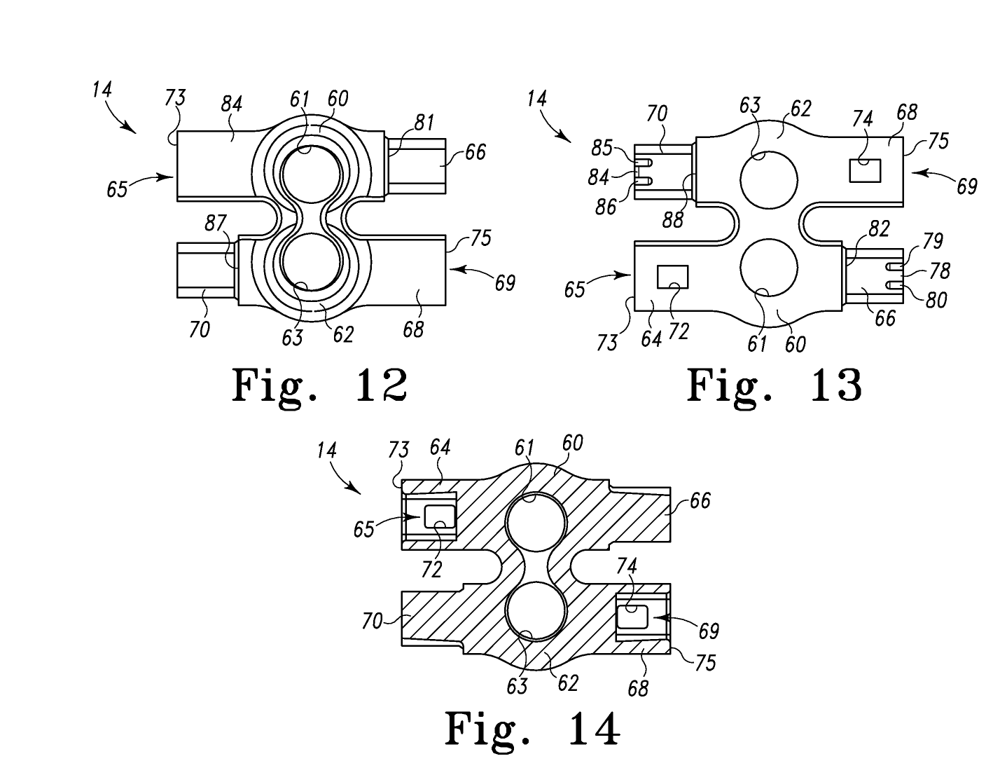

Referring to FIGS. 12-14 the center or middle plate component 14 will be described. The middle plate component 14 has a first boss 60 and a second boss 62. A bore 61 is provided in the first boss 60 and is configured to receive a bone screw (not shown) therethrough for attaching the center plate component 14 onto a vertebra. A bore 63 is provided in the second boss 62 and is configured to receive a bone screw (not shown) therethrough for attaching the center plate component 14 onto a vertebra. Extending from one side of the boss 60 is a leg 64 defining a socket 65. Extending from one side of the boss 62 is a leg 70 defining a projection. The legs 64 and 70 are on the same side of their respective bosses and provide an opening therebetween that defines a graft window. Extending from another side of the boss 60 opposite the leg 64 is a leg 66 defining a projection. Extending from another side of the boss 62 opposite the leg 70 is a leg 68 defining a socket 69. The legs 66 and 68 are on the same side of their respective bosses and provide an opening therebetween that defines a graft window.

The socket 65 of the leg 44 and the socket 69 are sized to receive the projection 48 of an end plate component 12 or one of the projections 66 and 70 of the center plate component such that when a multi level modular spine plate made of one or more central plate components 14 and two identical end plate components 12 are joined, the projection of one plate component (end or center) is received in the socket of another plate component (end or center) and the projection of the another end plate component (end or center) is received in the socket of the a plate component (end or center). The center plate component is fashioned such that one side of the bosses 60, 62 has a socket and projection while the other end has a socket and projection opposite to the other side of the bosses 60, 62. In this manner, a center plate component 14 may be joined, attached or connected to another center plate component 14 on either end thereof through a 180° rotation of a center plate component 14 relative to an adjacent center plate component 14. As well, an end plate component 12 may be joined, attached or connected to either end of a center plate component 14 through appropriate rotation of an end plate component 12 relative to the center plate component as necessary.

As best seen in FIGS. 13 and 14, the projection 66 has a resilient flange 78 on an end thereof formed in conjunction with side slots 79, 80 and middle slot (not seen) in like manner to that on the end component 12. The slots 79, 80 aid in defining the flange 78. The flange 78 is resilient in that it deforms into the middle slot when received in a socket of a plate component 12 or 14. The leg 68 has an opening or slot 74 within and in communication with the socket 75. The flange of a projection of an end plate component 12 or center plate component 14 is received in the slot 74 when an end plate component 12 or center plate component 14 is being engaged therewith. The flange thus resiliently snaps into the socket to provide a snap fit and coupling of a projection with a socket. The surfaces 81 and 82 of the projection 66 abut a socket end (such as socket end 73 of a center plate component 14).

The projection 70 has a resilient flange 84 on an end thereof formed in conjunction with side slots 85, 86 and middle slot (not seen) in like manner to that on the end component 12. The slots 85, 86 aid in defining the flange 84. The flange 84 is resilient in that it deforms into the middle slot when received in a socket of a plate component 12 or 14. The leg 64 has an opening or slot 72 within and in communication with the socket 65. The flange of a projection of an end plate component 12 or center plate component 14 is received in the slot 72 when an end plate component 12 or center plate component 14 is being engaged therewith. The flange thus resiliently snaps into the socket to provide a snap fit and coupling of a projection with a socket. The surfaces 87 and 88 of the projection 70 abut a socket end (such as socket end 75 of a center plate component 14).

It can be appreciated that with only two plate components as configured in the present invention, an N-level modular spine plate may be formed that connects, joins or attaches to its plate component mate via a snap fit structure. As well, the slots of the sockets may be configured to allow limited movement of the flange of a projection therein to provide limited movement of the plate components relative to one another.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A spine plate comprising:
a first spine plate component having a first spine plate component projection; and
a second spine plate component having a second spine plate component socket;
the first and second spine plate components joinable to form a spine plate wherein the first spine plate component projection is received in the second spine plate component socket.

2. The spine plate of claim 1, wherein:
the first spine plate component has a first spine plate component socket; and
the second spine plate component has a second spine plate projection;
the second spine plate component projection received in the first spine plate component socket when the first and second spine plate components are joined.

3. The spine plate of claim 2, wherein:
the first spine plate component includes a first spine plate component first boss with a first boss bone screw bore and a first spine plate component second boss with a second boss bone screw bore, the first spine plate component projection extending from the first spine plate component first boss and the first spine plate component socket extending from the first spine plate component second boss; and
the second spine plate component includes a second spine plate component first boss with a first boss bone screw bore and a second spine plate component second boss with a second boss bone screw bore, the second spine plate component socket extending from the second spine plate component first boss and the second spine plate component projection extending from the second spine plate component second boss.

4. The spine plate of claim 3, wherein the first and second spine plate components are identical and join to form the spine plate by rotation of one of the first and second spine plate components 180° relative to the other of the first and second spine plate components.

5. The spine plate of claim 2, wherein:
the first spine plate component projection includes a first resilient flange;
the first spine plate component socket includes a first slot;
the second spine plate component socket includes a second slot; and
the second spine plate component projection includes a second resilient flange;
the first resilient flange received in the second slot when the first spine plate component projection is received in the second spine plate projection socket and the second resilient flange is received in the first slot when the first and second spine plate components are joined thereby providing two snap fitted joints between the first and second spine plate components.

6. The spine plate of claim 5, wherein:
the first slot is sized to allow limited axial movement of the second resilient flange therein when the second spine plate component projection is received in the first spine plate component socket; and
the second slot is sized to allow limited axial movement of the first resilient flange therein when the first spine plate component projection is received in the second spine plate component socket;
whereby the first and second spine plate components exhibit limited axial movement between themselves when the first and second spine plate components are joined.

7. The spine plate of claim 1, wherein:
the first spine plate component projection includes a resilient flange; and
the second spine plate component socket includes a slot;
the resilient flange received in the slot when the first spine plate component projection is received in the second spine plate projection socket when the first and second spine plate components are joined thereby providing a snap fitted joint between the first and second spine plate components.

8. The spine plate of claim 7, wherein
the slot is sized to allow limited axial movement of the resilient flange therein when the first spine plate component projection is received in the second spine plate component socket;
whereby the first and second spine plate components exhibit limited axial movement between themselves when the first and second spine plate components are joined.

9. A modular spine plate kit for creating an N-level spine plate, the modular spine plate kit comprising:
a plurality of end spine plate components; and
a plurality of center spine plate components,
each one of the plurality of end spine plate components having an end plate projection extending from a side of a first end boss having a first end plate bone screw bore, and an end socket extending from a side of a second end boss having a second end plate bone screw bore, the end plate projection and the end plate socket being parallel to one another;
each one of the plurality of second spine plate components having a first projection extending from one side of a first center boss having a first center bone screw bore, a first socket extending from one side of a second center boss having a second center bone screw bore, a second projection extending from an opposite side of the first center boss, and a second projection extending from an opposite side of the second center boss, the first projection and the first socket being parallel to each other, and the second projection and the second socket being parallel to each other;
wherein a single level spine plate is formed joining two end spine plate components one of which is turned 180° relative to each other, the end plate sockets of which are sized to receive the end plate projections thereof, a two level spine plate is formed joining a first end spine plate component to one side of a center spine plate component and a second end spine plate component to another side of the center spine plate component, and a spine plate greater than three levels is formed by joining additional center plate components between the center plate component and an end plate component of a two level spine plate.

10. The modular spine plate kit of claim 9, wherein each one of the projections includes a resilient flange and each one of the sockets includes a slot configured to accept a resilient flange thereby providing a snap fit coupling of end and center spine plate components when projections are received in sockets of the spine plate components.

11. The modular spine plate kit of claim 10, wherein each slot is sized to allow limited axial movement of a received resilient flange therein when projections are received in sockets of the spine plate components whereby the spine plate components exhibit limited axial movement between themselves.

12. A modular spine plate comprising:
a first spine plate component having a first spine plate component projection and a first spine plate component socket, the first spine plate component projection and the first spine plate component socket parallel to one another; and
a second spine plate component having a second spine plate component socket and a second spine plate component socket;
the first and second spine plate components joinable to form a spine plate wherein the first spine plate component projection is received in the second spine plate component socket and the second spine plate component projection is received in the first spine plate component socket.

13. The modular spine plate of claim 12, wherein the projections each include a resilient flange and the sockets each include a slot configured to receive a resilient flange to provide a snap fit coupling of the first and second spine plate components when the first and second spine plate components are joined.

14. The modular spine plate of claim 13, wherein the slots are sized to allow limited axial movement of the resilient flanges therein whereby the joined spine plate components exhibit limited axial movement therebetween.

15. The modular spine plate of claim 12, wherein the first and second spine plate components are identical and join to form a one-level spine plate by rotation of one of the first and second spine plate components 180° relative to the other of the first and second spine plate components.

16. The modular spine plate of claim 12, further comprising:
a third spine plate component having a first projection extending from one side of a first center boss, a first socket extending from one side of a second center boss, a second projection extending from an opposite side of the first center boss, and a second projection extending from an opposite side of the second center boss, the first projection and the first socket being parallel to each other, and the second projection and the second socket being parallel to each other;
wherein the third spine plate component is joinable between the first and second spine plate components by reception of its projections with the sockets of the first and second spine plate components and the reception of the projections of the first and second spine plate components with the sockets of the third spine plate component to thereby form a two-level spine plate.

17. The modular spine plate of claim 16, wherein additional third spine plate components are connected between a third spine plate component and a first or second spine plate component to thereby form an N-level spine plate.

18. The modular spine plate of claim 17, wherein each projection includes a resilient flange and each socket includes a slot configured to receive a resilient flange to provide a snap fit coupling of the spine plate components when the spine plate components are joined.
